(19) United States
(12) Patent Application Publication
(10) Pub. No.: US 2022/0111111 A1
(43) Pub. Date: Apr. 14, 2022

(54) COMPOSITION FOR USE IN WOUND DRESSINGS

(71) Applicant: SCAPA TAPES NA, Windsor, CT (US)

(72) Inventors: Anne WIBAUX, Dunstable (GB); Faisal ALI, Orangeburg, NY (US)

(73) Assignee: SCAPA TAPES NA, Windsor, CT (US)

(21) Appl. No.: 17/423,553

(22) PCT Filed: Jan. 17, 2020
(86) PCT No.: PCT/US2020/013991

§ 371 Date: Jul. 16, 2021

Related U.S. Application Data
(60) Provisional application No. 62/793,659, filed on Jan. 17, 2019.

(51) Int. Cl. A61L 15/22; A61L 15/58; A61L 24/04

(52) U.S. Cl. CPC A61L 15/225; A61L 2400/14; A61L 24/043; A61L 15/58

(57) ABSTRACT

A composition comprising an acrylic adhesive, a thermoplastic absorbent polymer, and a gelling agent, a wound dressing comprising said composition, methods of making said composition and wound dressing, and the uses of said composition and wound dressings for treatment of wounds and/or for securement of devices to the skin.

### CROSS-REFERENCE TO RELATED APPLICATIONS

This application claims the benefit of priority to U.S. Provisional Application No. 62/793,659, filed on Jan. 17, 2019, which is incorporated by reference herein in its entirety.

### TECHNICAL FIELD

The present invention relates generally to compositions that have good adhesive properties on human skin, good fluid handling capacity, low trauma upon removal, and repositionability. The present invention further relates to wound dressings comprising said compositions, and the various uses of said compositions and wound dressings, for example for treating or preventing a wound and/or for securing a device to the skin of the patient. The present invention also relates to methods of making said compositions and wound dressings.

### BACKGROUND

The production of exudate or fluid from a mammalian wound is important for wound healing. Exudate is an acidic solution containing mainly water, but may also contain electrolytes, nutrients, proteins, inflammatory mediators, protein digesting enzymes, growth factors and various types of cell (e.g. white blood cells). Whilst wound healing generally benefits from a moist environment, too much exudate may delay healing and deplete patient quality of life.

Wound dressings may be designed to assist in maintaining a moist wound environment and/or managing exudate accumulation in the wound and pen-wound area. For example, wound dressings may be designed to absorb excess wound exudate and/or allow transmission of water vapour through the dressing.

Hydrocolloid adhesives, which may be made of a continuous rubbery phase with a discontinuous gelling phase, are medically useful pressure sensitive adhesives, and are useful as wound dressings because they are both absorbent and adhesive. However, hydrocolloids may be painful and/or damage the fragile peri-wound area since they adhere to skin and are retained on the skin under a range of adverse conditions (including contact with water). Hydrocolloids also have a low moisture vapour transmission rate, which limits their use on infected wounds as well as their capacity to manage large amounts of exudate.

It is therefore desirable to provide alternative or improved compositions that are highly absorbent, have a good moisture vapour transmission rate, and are adhesive to the skin, but provide repositionability and reduced pain upon removal.

### SUMMARY

In accordance with a first aspect of the present invention there is provided a composition comprising an acrylic adhesive, a thermoplastic absorbent polymer, and a gelling agent.

In accordance with a second aspect of the present invention there is provided a wound dressing comprising a composition according to the first aspect of the present invention, including any embodiment thereof.

In accordance with a third aspect of the present invention there is provided a method of making a composition according to the first aspect of the present invention, the method comprising mixing the acrylic adhesive, the thermoplastic absorbent polymer, and the gelling agent.

In accordance with a fourth aspect of the present invention there is provided a method of making a wound dressing according to the second aspect of the present invention, including any embodiment thereof.

In accordance with a fifth aspect of the present invention there is provided a composition according to the first aspect of the present invention or a wound dressing according to the second aspect of the present invention for use in treating or preventing a wound.

In accordance with a sixth aspect of the present invention there is provided a composition according to the first aspect of the present invention or a wound dressing according to the second aspect of the present invention for use in securing a device to skin.

In accordance with a seventh aspect of the present invention there is provided a use of a composition according to the first aspect of the present invention or a wound dressing according to a second aspect of the present invention for treating or preventing a wound.

In accordance with an eighth aspect of the present invention there is provided a use of a composition according to the first aspect of the present invention or a wound dressing according to a second aspect of the present invention for securing a device to skin.

In accordance with a ninth aspect of the present invention there is provided a method of treating a wound, the method comprising applying a composition according to the first aspect of the present invention or a wound dressing according to the second aspect of the present invention to the wound.

In accordance with a tenth aspect of the present invention there is provided a method of securing a device to skin, the method comprising applying a composition according to the first aspect of the present invention or a wound dressing according to the second aspect of the present invention to the device and/or skin where the device is to be secured.

In certain embodiments of any aspect of the present invention, the acrylic adhesive is an acrylic hot melt adhesive.

In certain embodiments of any aspect of the present invention, the composition comprises at least about 40 wt % of the acrylic adhesive. In certain embodiments of any aspect of the present invention, the composition comprises up to about 98 wt % of the acrylic adhesive. In certain embodiments of any aspect of the present invention, the composition comprises from about 40 wt % to about 98 wt % of the acrylic adhesive, for example from about 60 wt % to about 80 wt % of the acrylic adhesive.

In certain embodiments of any aspect of the present invention, the composition comprises at least about 1 wt % of the thermoplastic absorbent polymer. In certain embodiments of any aspect of the present invention, the composition comprises up to about 20 wt % of the thermoplastic absorbent polymer. In certain embodiments of any aspect of the present invention, the composition comprises from about 1 wt % to about 20 wt % of a thermoplastic absorbent polymer, for example from about 1 wt % to about 10 wt % of a thermoplastic absorbent polymer. In certain embodiments, the thermoplastic absorbent polymer absorbs water in an amount at least about 100% of its own weight. In certain embodiments, the thermoplastic absorbent polymer has a glass transition temperature (Tg) equal to or less than about 0° C. In certain embodiments, the thermoplastic absorbent polymer has a melting temperature equal to or less than about 120° C. In certain embodiments, the thermoplastic absorbent polymer is a polyurethane polymer.

In certain embodiments of any aspect of the present invention, the composition comprises at least about 1 wt % of a gelling agent. In certain embodiments of any aspect of the present invention, the composition comprises up to about 50 wt % of a gelling agent. In certain embodiments of any aspect of the present invention, the composition comprises from about 1 wt % to about 50 wt % of a gelling agent, for example from about 15 wt % to about 30 wt % of a gelling agent. In certain embodiments, the gelling agent is a superabsorbent polymer or a hydrocolloid. In certain embodiments, the gelling agent is carboxymethylcellulose, for example sodium carboxymethylcellulose.

In certain embodiments of any aspect of the present invention, the composition further comprises a polyol such as glycerol. In certain embodiments, the composition further comprises an active agent. In certain embodiments, the composition further comprises fibres such as polyethylene fibres. In certain embodiments, the composition has a thickness ranging from about 100 μm to about 2000 μm, for example from about 200 μm to about 500 μm.

In certain embodiments, the wound is a chronic wound or an acute wound.

Certain embodiments of any aspect of the present invention may provide one or more of the following advantages:
good absorption capacity;
good moisture vapour transmission rate;
good adhesion to the skin;
low trauma/pain upon removal from the skin; and
repositionability.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

There is provided herein a composition that provides good adhesion to the skin, good fluid absorption capacity, and good moisture vapour transmission rate whilst also reducing pain and/or trauma upon removal and enabling repositionability. There is further provided herein a wound dressing comprising a composition described herein.

The compositions provided herein comprise an acrylic adhesive, a thermoplastic absorbent polymer, and a gelling agent. In certain embodiments, the compositions provided herein consist essentially of or consist of an acrylic adhesive, a thermoplastic absorbent polymer, and a gelling agent. In certain embodiments, the compositions provided herein consist essentially of or consist of an acrylic adhesive, a thermoplastic absorbent polymer, a gelling agent, and optionally one or more polyols, optionally one or more active agents, and optionally one or more fibres. The acrylic adhesive may, for example, be an acrylic hot melt adhesive.

The compositions provided herein may, for example, comprise at least about 40 wt % of an acrylic adhesive, at least about 1 wt % of a thermoplastic absorbent polymer, and at least about 1 wt % of a gelling agent. The acrylic adhesive may, for example, be an acrylic hot melt adhesive.

The compositions described herein may, for example, have an absorption capacity of at least about 1 g/10 cm²/₂/4 hours. For example, the compositions may have an absorption capacity of at least about 1.5 g/10 cm²/24 hours or at least about 2 g/10 cm²/24 hours or at least about 2.5 g/10 cm²/24 hours or at least about 3 g/10 cm²/24 hours or at least about 3.5 g/10 cm²/24 hours or at least about 4 g/10 cm²/24 hours or at least about 4.5 g/10 cm²/24 hours or at least about 5 g/10 cm²/24 hours. For example, the compositions may have an absorption capacity up to about 10 g/10 cm²/24 hours or up to about 9.5 g/10 cm²/24 hours or up to about 9 g/10 cm²/24 hours or up to about 8.5 g/10 cm²/24 hours or up to about 8 g/10 cm²/24 hours or up to about 7.5 g/10 cm²/24 hours or up to about 7 g/10 cm²/24 hours or up to about 6.5 g/10 cm²/24 hours. For example, the compositions may have an absorption capacity ranging from about 1 g/10 cm²/24 hours to about 10 g/10 cm²/24 hours or from about 3 g/10 cm²/24 hours to about 8 g/10 cm²/24 hours or from about 4 g/10 cm²/24 hours to about 7.5 g/10 cm²/24 hours or from about 4.5 g/10 cm²/24 hours to about 7 g/10 cm²/24 hours. The absorption capacity of the compositions may be measured on samples having a thickness of 0.3 mm.

The compositions described herein may, for example, have a moisture vapour transmission rate (MVTR) of at least about 400 g/m²/24 hours. For example, the compositions may have a MVTR of at least about 450 g/m²/24 hours or at least about 500 g/m²/24 hours or at least about 550 g/m²/24 hours or at least about 600 g/m²/24 hours. For example, the compositions may have a MVTR up to about 15,000 g/m²/24 hours or up to about 1500 g/m²/24 hours or up to about 1200 g/m²/24 hours or up to about 1000 g/m²/24 hours. For example, the compositions may have a MVTR ranging from about 400 g/m²/24 hours to about 2000 g/m²/24 hours or from about 450 g/m²/24 hours to about 1500 g/m²/24 hours or from about 500 g/m²/24 hours to about 1200 g/m²/24 hours. The MVTR of the compositions may be measured when the composition is laminated onto a carrier, for example a polyurethane carrier such as Bioflex® Rx1281U or a Covestro® LPT9147T or a Covestro® LPT9147TX carrier.

Absorption capacity and MVTR may be measured in accordance with ISO 13726, for example as described below in the Examples.

The compositions described herein may, for example, have a tack of at least about 120 g. For example, the compositions may have a tack of at least about 125 g or at least about 130 g or at least about 135 g or at least about 140 g or at least about 145 g or at least about 150 g or at least about 155 g or at least about 160 g or at least about 165 g or at least about 170 g. For example, the compositions may have a tack up to about 250 g or up to about 240 g or up to about 230 g or up to about 220 g or up to about 210 g or up to about 200 g or up to about 190 g or up to about 180 g. For example, the compositions may have a tack ranging from about 150 g to about 250 g or from about 155 g to about 220 g or from about 160 g to about 200 g or from about 160 g to about 190 g.

Tack is the force required to separate the adhesive from a surface after they have been brought into contact under a load equal to the weight of the composition. Tack may be measured using a calibrated PT-1000 Probe Tack Tester, for example as described below in the examples.

The compositions described herein may, for example, have a peel of at least about 1 N. For example, the compositions may have a peel of at least about 1.2 N or at least about 1.4 N or at least about 1.5 N or at least about 1.8 N or at least about 2 N. For example, the compositions may have a peel up to about 7 N or up to about 6.5 N or up to about 6 N or up to about 5.5 N or up to about 5 N. For example, the compositions may have a peel up to about 4 N or up to about 3.8 N or up to about 3.5 N or up to about 3.2 N or up to about 3 N. For example, the compositions may have a peel ranging from about 1 N to about 7 N or from about 1.5 N to about 6.5 N or from about 2 N to about 5 N or from about 1 N to about 4 N or from about 1.5 N to about 3.5 N or from about 2 N to about 3 N.

Peel is the force required to remove a composition from a stainless steel 304 test panel at a 90° angle at a standard rate of 5.0 mm/s, in accordance with ASTM A 666. Peel may, for example, be measured as described below in the examples.

The composition may, for example, be free of ingredients that are not compatible with mammalian skin, for example ingredients that cause irritation or toxicity to mammals. For example, the composition may be free of colophonium derivatives or natural rubber. The composition may, for example, be a medical grade composition.

The composition may, for example, have a thickness of at least about 100 μm. For example, the composition may have a thickness of at least about 150 μm or at least about 200 μm or at least about 300 μm. For example, the composition may have a thickness up to about 2000 pm, for example up to about 1800 μm or up to about 1600 μm or up to about 1500 μm or up to about 1400 μm or up to about 1200 μm or up to about 1000 μm or up to about 800 μm or up to about 600 μm or up to about 500 μm or up to about 400 μm. For example, the composition may have a thickness ranging from about 100 μm to about 2000 μm or from about 150 μm to about 1500 μm or from about 200 μm to about 1000 μm or from about 200 pm to about 500 μm.

### Acrylic Adhesive

The compositions described herein comprise an acrylic adhesive. In certain embodiments, the compositions described herein may comprise a mixture of two or more acrylic adhesives. One or more of the acrylic adhesives used in the compositions described herein may be an acrylic hot melt adhesive. For example, all of the acrylic adhesives used in the compositions described herein may be acrylic hot melt adhesives.

An acrylic adhesive is a thermoplastic material that includes acrylate monomers (derivatives of acrylic acid including, for example, acrylic acid, acrylic esters such as methyl methacrylate, methyl acrylate, and ethyl acrylate, and acrylonitrile).

An acrylic hot melt adhesive is a thermoplastic material that includes acrylate monomers that is solid at room temperature but which, when applied in a liquid (melted) state, sticks to a surface as it cools below its melting point.

The compositions described herein may, for example, comprise a continuous phase of an acrylic adhesive. For example, the compositions described herein may comprise a continuous phase of an acrylic hot melt adhesive.

Useful acrylate polymers and copolymers may or may not be self-crosslinking and may, for example, be formed from monomers chosen from: (1) hydroxyalkyl esters of acrylic or methacrylic acid in which the alkyl group comprises 2 to 4 carbon atoms, such as 2-hydroxyethyl acrylate, 2-hydroxyethyl methacrylate, 2-hydroxypropyl acrylate and 2-hydroxypropyl methacrylate; (2) alkyl esters of acrylic or methacrylic acid in which the alkyl group of the ester comprises 4 to 18 carbon atoms, such as n-butyl acrylate or methacrylate, isopropyl acrylate or methacrylate, n-hexyl methacrylate and 2-ethylhexyl acrylate; (3) α,β-unsaturated monocarboxylic or dicarboxylic acids, their anhydrides and their alkyl or alkenyl esters in which the alkyl group contains from 1 to 3 carbon atoms and the alkenyl group contains from 2 to 5 carbon atoms, such as acrylic acid, itaconic acid, maleic acid, maleic anhydride, alkyl methacrylate and the diethyl esters of fumaric or maleic acid; (4) vinyl monomers, such as vinyl acetate, acrylonitrile, vinyl propionate, vinylpyrrolidone and styrene; (5) monomers containing a functional group selected from amino, amino and epoxy groups, for example, acrylamide, N-butylacrylamide, alkylaminoalkyl and aminoalkyl derivatives of acrylic or methacrylic acid, such as amino-ethyl acrylate, aminoethyl methacrylate and 2-(dimethylamino) ethyl methacrylate, glycidyl methacrylate and glycidyl acrylate; (6) alkoxyalkyl esters of acrylic or methacrylic acid, for example methoxyethyl acrylates or methacrylates, butoxyethyl acrylates or methacrylates, methoxypropylene glycol acrylates or methacrylates and methoxypolyethylene glycol acrylates or methacrylates; and (7) hexamethylene glycol dimethacrylate.

The acrylic polymers may also contain a crosslinking agent.

The acrylic hot melt adhesive may, for example, be obtainable from Artimelt Inc., Tucker, Ga., USA, for example one or more of Artimelt™ M11-1506, Artimelt™ M11-1289, and Artimelt™ M11-1305.

The compositions described herein may comprise at least about 40 wt % of total acrylic adhesive. The compositions may, for example, comprise at least about 42 wt % or at least about 44 wt % or at least about 45 wt % or at least about 46 wt % or at least about 48 wt % or at least about 50 wt % or at least about 52 wt % or at least about 54 wt % or at least about 55 wt % or at least about 56 wt % or at least about 58 wt % or at least about 60 wt % or at least about 62 wt % or at least about 64 wt % or at least about 65 wt % or at least about 66 wt % or at least about 68 wt % or at least about 70 wt % of total acrylic adhesive. The compositions may, for example, comprise up to about 98 wt % of total acrylic adhesive. For example, the compositions may comprise up to about 96 wt % or up to about 95 wt % or up to about 94 wt % or up to about 92 wt % or up to about 90 wt % or up to about 88 wt % or up to about 86 wt % or up to about 85 wt % or up to about 84 wt % or up to about 82 wt % or up to about 80 wt % or up to about 78 wt % or up to about 76 wt % or up to about 75 wt % of total acrylic adhesive. For example, the compositions may comprise from about 40 wt % to about 98 wt % of total acrylic adhesive or from about 40 wt % to about 90 wt % or from about 50 wt % to about 85 wt % or from about 60 wt % to about 80 wt % or from about 50 wt % to about 80 wt % of total acrylic adhesive.

### Thermoplastic Absorbent Polymer

The compositions described herein also comprise a thermoplastic absorbent polymer. Thermoplastic polymers are polymers that become plastic on heating and harden upon cooling, and are able to repeat these processes. By “absorbent” polymer, it is meant that the polymer can absorb at least about 100% of its own weight in water.

The thermoplastic absorbent polymer may, for example, be able to absorb at least about 200% of its own weight in water. For example, the thermoplastic absorbent polymer may be able to absorb at least about 300% or at least about 400% or at least about 500% or at least about 600% or at least about 700% or at least 800% or at least about 900% of its own weight in water. For example, the thermoplastic absorbent polymer may comprise up to about 2000% or up to about 1800% or up to about 1600% or up to about 1400% or up to about 1200% or up to about 1000% of its weight in water. For example, the thermoplastic absorbent polymer may be able to absorb from about 200% to about 2000% or from about 500% to about 1500% or from about 800% to about 1200% of its weight in water. This may be measured in accordance with ASTM D570.

The thermoplastic absorbent polymer may, for example, have a glass transition temperature (Tg) equal to or less than about 0° C. For example, the thermoplastic absorbent polymer may have a Tg equal to or less than about −5° C. or equal to or less than about −10° C. or equal to or less than about −15° C. or equal to or less than about −20° C. or equal to or less than about −25° C. or equal to or less than about −30° C. or equal to or less than about −35° C. or equal to or less than about −40° C. or equal to or less than about −45° C. or equal to or less than about −50° C. or equal to or less than about −55° C. or equal to or less than about −60° C. or equal to or less than about −65° C. or equal to or less than about −70° C. or equal to or less than about −75° C. or equal to or less than about −80° C. or equal to or less than about −85° C. or equal to or less than about −90° C. or equal to or less than about −95° C. For example, the thermoplastic absorbent polymer may have a Tg equal to or greater than about −150° C. or equal to or greater than about −145° C. or equal to or greater than about −140° C. or equal to or greater than about −135° C. or equal to or greater than about −130° C. or equal to or greater than about −125° C. or equal to or greater than about −120° C. or equal to or greater than about −115° C. or equal to or greater than about −110° C. or equal to or greater than about −105° C. or equal to or greater than about −100° C. or equal to or greater than about −95° C. or equal to or greater than about −90° C. or equal to or greater than about −85° C. or equal to or greater than about −80° C. or equal to or greater than about −75° C. For example, the thermoplastic absorbent polymer may have a Tg ranging from about −150° C. to about 0° C. or from about −100° C. to about −25° C. or from about −75° C. to about −35° C. Tg may be measured in accordance with ISO 11357-2.

The thermoplastic absorbent polymer may, for example, have a melting temperature equal to or less than about 120° C. For example, the thermoplastic absorbent polymer may have a melting temperature equal to or less than about 110° C. or equal to or less than about 100° C. or equal to or less than about 90° C. or equal to or less than about 80° C. or equal to or less than about 70° C. or equal to or less than about 60° C. The thermoplastic absorbent polymer may, for example, have a melting temperature equal to or greater than about 30° C. For example, the thermoplastic absorbent polymer may have a melting temperature equal to or greater than about 40° C. or equal to or greater than about 50° C. or equal to or greater than about 60° C. For example, the thermoplastic absorbent polymer may have a melting temperature ranging from about 30° C. to about 120° C. or from about 40° C. to about 100° C. or from about 40° C. to about 80° C. The melting temperature may, for example, be measured in accordance with ISO 11357-2.

The thermoplastic absorbent polymer may, for example, have a melt flow index of at least about 1 g/10 minutes. For example, the thermoplastic absorbent polymer may have a melt flow index of at least about 2 g/10 minutes or at least about 3 g/10 minutes or at least about 4 g/10 minutes or at least about 5 g/10 minutes. The thermoplastic absorbent polymer may, for example, have a melt flow index up to about 50 g/10 minutes. For example, the thermoplastic absorbent polymer may have a melt flow index up to about 45 g/10 minutes or up to about 40 g/10 minutes or up to about 35 g/10 minutes or up to about 30 g!10 minutes or up to about 25 g/10 minutes or up to about 20 g/10 minutes. For example, the thermoplastic absorbent polymer may have a melt flow index ranging from about 1 g/10 minutes to about 50 g/10 minutes or from about 2 g/10 minutes to about 30 g/10 minutes or from about 5 g!10 minutes to about 25 g!10 minutes. Melt flow index may, for example, be measured in accordance with ASTM D1238-1700/2.

The thermoplastic absorbent polymer may, for example, be selected from polyethers (e.g. polyurethanes), polyamides, polyesters, polyureas, polyethylene oxide, polysaccharide, polymaleic anhydride, polyethylene oxide/polypropylene oxide copolymers, and combinations thereof.

The thermoplastic absorbent polymer may, for example, be a polyether polymer (a polymer including more than one ether linkages). The polyether polymer may, for example, be an aliphatic polyether polymer.

The thermoplastic absorbent polymer may, for example, be a polyurethane polymer (a polymer including organic units joined by carbamate (urethane) links. Polyurethane polymers may be made by reacting a di- or tri-isocyanate with a polyol. The polyurethane polymer may, for example, be an aliphatic polyurethane polymer.

The thermoplastic absorbent polymer may, for example, be obtainable from Lubrizol, for example a Tecophilic™ polymer such as Tecophilic™ TG-500 or Tecophilic™ TG-2000.

The thermoplastic absorbent polymer may be present in the composition in an amount of at least about 1 wt %. For example, the thermoplastic absorbent polymer may be present in the composition in an amount of at least about 1.5 wt % or at least about 2 wt % or at least about 2.5 wt % or at least about 3 wt %. The thermoplastic absorbent polymer may, for example, be present in the composition in an amount up to about 20 wt %. For example, the thermoplastic absorbent polymer may be present in the composition in an amount up to about 18 wt % or up to about 16 wt % or up to about 15 wt % or up to about 14 wt % or up to about 12 wt % or up to about 10 wt % or up to about 8 wt % or up to about 6 wt % or up to about 5 wt %, For example, the composition may comprise from about 1 wt % to about 20 wt % of the thermoplastic absorbent polymer, for example from about 1.5 wt % to about 15 wt % or from about 2 wt % to about 10 wt % or from about 2 wt % to about 5 wt % of the thermoplastic absorbent polymer.

### Gelling Agent

The compositions described herein also comprise a gelling agent. A gelling agent is a material that forms a gel upon contact with water, dissolving in the liquid phase to form a colloid mixture.

The compositions described herein may, for example, comprise a discontinuous phase of a gelling agent.

The gelling agent may, for example, be a superabsorbent polymeror a hydrocolloid.

A superabsorbent polymer is a water-swellable, hydrogel-forming absorbent polymer capable of absorbing large quantities of liquids such as water, body fluids (e.g. urine, blood), and the like. The superabsorbent polymer may be capable of retaining the absorbed fluids under moderate pressures. The superabsorbent polymer may not dissolve in water and/or saline. Increasing the degree of crosslinking may increase the superabsorbent polymer's total fluid holding capacity under load. The degree of crosslinking is preferably optimized to obtain a composition in which the rate and amount of absorbency are optimized. The superabsorbent polymers may, for example, be at least 10%, for example from about 10% to about 50%, for example from about 20% to 40% crosslinked.

The superabsorbent polymer may, for example, absorb at least about 2 times its own weight in water. For example, the superabsorbent polymer may absorb at least about 3 times or at least about 5 times or at least about 10 times or at least about 15 times or at least about 20 times or at least about 25 times or at least about 30 times or at least about 35 times or at least about 40 times or at least about 45 times or at least about 50 times its own weight in water. For example, the superabsorbent polymer may absorb up to about 1000 times or up to about 800 times or up to about 600 times or up to about 500 times or up to about 400 times or up to about 300 times or up to about 200 times or up to about 100 times its own weight in water. For example, the superabsorbent polymer may absorb from about 2 times to about 1000 times or form about 3 times to about 600 times or from about 5 times to about 400 times or from about 10 times to about 200 times its own weight in water. The superabsorbent polymer may, for example, absorb at least about 2 times or at least about 3 times or at least about 5 times or at least about 10 times or at least about 20 times or at least about 50 times its own weight in saline. For example, the superabsorbent polymer may absorb up to about 1000 times or up to about 800 times or up to about 600 times or up to about 500 times or up to about 400 times or up to about 200 times or up to about 100 times its own weight in saline. For example, the superabsorbent polymer may absorb from about 2 times to about 1000 times or from about 5 times to about 600 times or from about 10 times to about 400 times or from about 10 times to about 200 times its own weight in saline. This may be measured by the tea bag test method.

Examples of superabsorbent polymers include crosslinked and/or polymerized α,β-beta ethylenically unsaturated mono- and dicarboxylic acids and acid anhydride monomers including, for example, acrylic acid, methacrylic acid, crotonic acid, maleic acid/anhydride, itaconic acid, fumaric acid, and combinations thereof. The superabsorbent polymer may, for example, comprise polyacrylic acid (e.g. sodium polyacrylate), polyacrylamide copolymer, ethylene maleic anhydride copolymer, polyvinyl alcohol copolymers, polyethylene oxide, or starch grafted copolymer of polyacrylonitrile. Super absorbent polymers include, for example, crosslinked acrylate polymers, crosslinked products of vinyl alcohol-acrylate copolymers, crosslinked products of polyvinyl alcohols grafted with maleic anhydride, cross-linked products of acrylate-methacrylate copolymers, crosslinked saponification products of methyl acrylate-vinyl acetate copolymers, crosslinked products of starch acrylate graft copolymers, crosslinked saponification products of starch acrylonitrile graft copolymers, crosslinked products of carboxymethyl cellulose polymers and crosslinked products of isobutylene-maleic anhydride copolymers, acrylonitrile polymer grafted onto the backbone of starch molecules and combinations thereof.

The superabsorbent polymer may be present in the composition as particles, for example spherical particles. The superabsorbent particles may have an average particle size of from about 1 μm to about 400 μm, Preferably the particles have an average particle size of from about 20 μm to about 200 μm, and more preferably from 20 μm to 150 μm. In one embodiment, the particle size of the particles is less than 150 μm, or less than 100 μm. Particle size may, for example be measured by laser diffraction.

A hydrocolloid may be a water absorbing and/or water swellable material. The hydrocolloid may be synthetic or naturally-occurring. The hydrocolloid may, for example, be crosslinked. The hydrocolloid may, for example, be selected from polyvinyl alcohol, polyvinyl acetate, polyvinyl pyrollidone, polyacrylic acid, polyhydroxyalkyl acrylates, polyacrylamides, high molecular weight polyethylene glycols polypropylene glycols, carboxymethylcellulose (CMC) (e.g. sodium carboxmethylcellulose, cross-linked sodium carboxymethylcellulose, and/or crystalline carboxymethylcellulose), pectin, gelatin, high molecular weight carbowax, carboxypolymethylene, carboxymethyl starches, alginates, carrageenan, gelatine, citrus pectin, powdered pectin, synthetic or natural gums such as guar gum, gum Arabic, locust bean gum, tragacanth gum, dextran, starches, karaya, starch-acrylonitrile graft copolymer and mixtures thereof. Preferred hydrocolloids may be carboxymethylcellulose (CM), alginates, and pectin.

The hydrocolloid may be particles, for example having an average particle size of from about 1 μm to about 400 μm, for example from about 20 μm to about 200 μm, for example from 20 μm to 150 μm. For example, the particle size of the particles may be less than about 150 μm, or less than about 100 μm. Particle size may, for example, be measured by laser diffraction.

The gelling agent may, for example, be carboxymethylcellulose, for example sodium carboxymethylcel lulose.

The gelling agent may be present in the compositions described herein in an amount of at least about 1 wt %. For example, the compositions may comprise at least about 2 wt % or at least about 4 wt % or at least about 5 wt % or at least about 6 wt % or at least about 8 wt % or at least about 10 wt % or at least about 12 wt % or at least about 14 wt % or at least about 15 wt % of the gelling agent. For example, the compositions may comprise up to about 50 wt % of the gelling agent, for example up to about 48 wt % or up to about 46 wt % or up to about 45 wt % or up to about 44 wt % or up to about 42 wt % or up to about 40 wt % or up to about 38 wt % or up to about 36 wt % or up to about 35 wt % or up to about 34 wt % or up to about 32 wt % or up to about 30 wt % of the gelling agent. For example, the gelling agent may be present in the composition in an amount ranging from about 1 wt % to about 50 wt % or from about 2 wt % to about 40 wt % or from about 5 wt % to about 35 wt % or from about 10 wt % to about 30 wt %.

### Optional Additives

The composition may include one or more additional ingredients. Such additional ingredients may be selected from additives, including, for example, water, organic plasticisers, surfactants, polymeric material (hydrophobic or hydrophilic in nature, including proteins, enzymes, naturally occurring polymers and gums), synthetic polymers with and without pendant carboxylic acids, electrolytes, pH regulators, colorants, chloride sources, bioactive compounds and mixtures thereof. The polymers can be natural polymers (e.g. xanthan gum), synthetic polymers (e.g. polyoxypropylene-polyoxyethylene block copolymer or poly-(methyl vinyl ether maleic anhydride)), or any combination thereof.

The composition described herein may comprise up to about 20 wt % of optional further additives, for example up to about 15 wt % or up to about 10 wt % or up to about 5 wt %.

The compositions described herein may comprise one or more further additives.

For example, the compositions may comprise one or more polyols such as, for example, glycol, sorbitol, mannitol, maltitol, lactitol, xylitol, and combinations thereof.

The composition may, for example, comprise up to about 20 wt % of total polyols. For example, the composition may comprise up to about 18 wt % or up to about 16 wt % or up to about 15 wt % or up to about 14 wt % or up to about 12 wt % or up to about 10 wt % or up to about 8 wt % or up to about 6 wt % or up to about 5 wt % of total polyols. For example, the composition may comprise at least about 1 wt % total polyols. For example, the composition may comprise at least about 2 wt % or at least about 3 wt % or at least about 4 wt % or at least about 5 wt % total polyols.

For example, the compositions may comprise one or more active agents (any compound or mixture included within the composition for some effect it has on living systems, whether the living system be bacteria or other microorganisms or higher animals such as the patient). Examples of active agents include, for example, pharmaceutically active agents, agents for stimulating the healing of wounds and/or for restricting or preventing scarring (e.g. growth factors e.g. from GroPep Ltd, Australia or Procyte, USA (see, e.g. WO-A-98/02270, the contents of which are incorporated herein by reference); cell nutrients (see, e.g., WO-A-93/04691 the contents of which are incorporated herein by reference); glucose (see, e.g., WO-A-93/10795, the contents of which are incorporated herein by reference); an anabolic hormone or hormone mixture such as insulin, triiodothyronine, thyroxine or any combination thereof (see, e.g., WO-A−93/04691 , the contents of which are incorporated herein by reference); or any combination thereof), circulatory drugs (e.g. organic nitrates, beta blockers, ACE inhibitors; alpha-2 blockers), antimicrobial agents (e.g. sauces of oxygen and/or iodine (e.g. hydrogen peroxide or a source thereof and/or an iodide salt such as potassium iodide) (see, for example BioxzymeTM technology, for example in The Sunday Telegraph (UK) 26 Jan. 2003 or the discussion of the Oxyzyme™ system at www.wounds-uk.com/posterabstracfs2003.pdf); honey (e.g. active Manuka honey); antimicrobial metals, metal ions and salts, such as, for example, silver-containing antimicrobial agents (e.g. colloidal silver, silver oxide, silver nitrate, silver thiosulphate, silver sulphadiazine, or any combination thereof); or any combination thereof), antiseptic agents, antibiotics, androgenic steroids (e.g. testosterone, methyl testosterone, fluoximesterone), oestrogens, progestagenic hormones (e.g. progesterone), drugs acting on the nervous system (e.g. sedatives, hypnotics, anxiolytics, antidepressants, analgesics, anaesthetics), anti-inflammatory agents (e.g. hydrocortisone, cortisone, dexamethasone, triamcinolone, prednisolone, ibuprofen, naproxen, fenoprofen, flurbiprofen, indoprofen, ketoprofen, piroxicam, diflunisal), local anesthetic (e.g. lidocaine, prilocaine, procaine, benzocaine), antioxidants, antihistamines, respiratory drugs (e.g. theophylline, beta2-adrenergic agonists), sympathicomimetics (e.g dopamine, phenylpropanolamine, phenylephrine), antimuscarines (atropine, scopolamine, homatropine, benzatropine), prostaglandins, antioestrogens (e.g. tamoxifen), antimigraine agents (e.g. dihydroergotamine, pizotyline), antiulcer agents (e.g. misoprostol, omeprazole, enprostil, ranitidine), vitamins (e.g. vitamins A, D, E, K, B, C), minerals (calcium, chromium, selenium, iron, manganese, magnesium, potassium, sodium, phosphorous, zinc, copper), botanicals, and combinations thereof.

The composition may, for example, comprise up to about 10 wt % total active agents. For example, the composition may comprise up to about 9 wt % or up to about 8 wt % or up to about 7 wt % or up to about 6 wt % or up to about 5 wt % or up to about 4 wt % or up to about 3 wt % total active agents. For example, the composition may comprise from about 0 wt % to about 10 wt % or from about 0.5 wt % to about 8 wt % or from about 1 wt % to about 5 wt % of total active agents.

For example, the compositions may further comprise fibres, for example polymer fibres such as polyethylene fibres, polypropylene fibres, cotton fibres, and combinations thereof.

The fibres may, for example, have a length of equal to or greater than about 0.05 mm, for example equal to or greater than about 0.1 mm or equal to or greater than about 0.5 mm. The fibres may, for example, have a length equal to or less than about 5 mm or equal to or less than about 4 mm or equal to or less than about 3 mm or equal to or less than about 2 mm. For example, the fibres may have a length ranging from about 0.05 mm to about 5 mm or from about 0.1 mm to about 3 mm or from about 0.1 mm to about 2 mm.

The fibres may, for example, have a surface area equal to or greater than about 1 m²/g or equal to or greater than about 2 m²/g or equal to or greater than about 3 m²/g or equal to or greater than about 4 m²/g or equal to or greater than about 5 m²/g. For example, the fibres may have a surface area equal to or less than about 30 m²/g or equal to or less than about 25 m²/g or equal to or less than about 20 m²/g or equal to or less than about 15 m²/g or equal to or less than about 10 m²/g. For example, the fibres may have a surface area ranging from about 1 m²/g to about 30 m²/g or from about 2 m²/g to about 25 m²/g or from about 5 m²/g to about 20 m²/g or from about 5 m²/g to about 10 m²/g. The surface area of the fibres may, for example, be measured by gas absorption.

The composition may, for example, comprise up to about 10 wt % of total fibres. For example, the composition may comprise up to about 9 wt % or up to about 8 wt % or up to about 7 wt % or up to about 6 wt % or up to about 5 wt % or up to about 4 wt % of total fibres. For example, the composition may comprise at least about 0.5 wt % of total fibres. For example, the composition may comprise at least about 1 wt % or at least about 2 wt % total fibres.

### Wound Dressing

There is further provided herein a wound dressing comprising a composition as described herein.

The wound dressing may, for example, be a multilayer composition. The composition described herein may, for example, form a discrete layer of the multilayer composition. Each of the layers in the multilayer composition may be adhered together using an adhesive composition. Some layers of the wound dressing may be inherently adhesive. The adhesive may, for example, be a medical grade adhesive. The adhesive may, for example, be a pressure sensitive adhesive. The adhesive may, for example, comprise one or more of acrylic, rubber, silicone, polyurethane or hybrid. The layer comprising the composition described herein may, for example, have a thickness of at least about 100 μm. For example, the discrete layer of the composition described herein may have a thickness of at least about 150 μm or at least about 200 μm or at least about 300 μm. For example, the layer comprising the composition described herein may have a thickness up to about 2000 μm, for example up to about 1800 μm or up to about 1600 μm or up to about 1500 μm or up to about 1400 μm or up to about 1200 μm or up to about 1000 μm or up to about 800 μm or up to about 600 μm or up to about 500 μm or up to about 400 μm. For example, the layer comprising the composition described herein may have a thickness ranging from about 100 μm to about 2000 μm or from about 150 μm to about 1500 μm or from about 200 μm to about 1000 μm or from about 200 μm to about 500 μm.

The wound dressing may, for example, comprise a breathable backing layer by which is meant a layer that allows the transmission of moisture at at least the same rate as the composition described herein. The breathable backing layer may, for example, have a moisture vapour transmission rate of at least about 1500 g/m²/24 hours, for example at least about 2000 g/m²/24 hours or at least about 3000 g/m²/24 hours or at least about 4000 g/m²/24 hours or at least about 5000 g/m²/24 hours. For example, the breathable backing layer may have a MVTR up to about 15,000 g/m²/24 hours or up to about 10,000 g/m²/24 hours or up to about 8000 g/m²/24 hours. For example, the backing layer may have a MVTR ranging from about 1500 g/m²/24 hours to about 15,000 g/m²/24 hours or from about 2000 g/m²/24 hours to about 10,000 g/m²/24 hours or from about 3000 g/m²/24 hours to about 8000 g/m²/24 hours.

The backing layer may, for example, be a polymeric elastic or flexible film. The backing layer may, for example, be impermeable to liquid and/or bacteria.

The backing layer may, for example, comprise polyurethane, polyolefins (e.g. polyester), elastomeric polyester, blends of polyurethane and polyester, polystyrene, polyvinyl chloride, polyetheramide block copolymer, porous polyethylene, carboxymethylcellulose, or copolymers or mixtures thereof. The backing layer may, for example, be a copolymer of polyether and polyurethane.

The backing layer may, for example, have a thickness of at least about 5 μm. For example, the backing layer may have a thickness of at least about 10 μm or at least about 15 μm or at least about 20 μm. For example, the backing layer may have a thickness up to about 500 μm or up to about 450 μm or up to about 400 μm or up to about 350 μm or up to about 300 μm or up to about 250 μm or up to about 200 μm or up to about 150 μm or up to about 100 μm or up to about 75 μm or up to about 50 μm or up to about 40 μm or up to about 30 μm. For example, the backing layer may have a thickness ranging from about 5 μm to about 500 μm or from about 15 μm to about 150 μm or from about 20 μm to about 100 μm or from about 20 μm to about 50 μm or from about 20 μm to about 30 μm.

The backing layer may, for example, be located on the outer-facing side of the composition described herein. The backing layer may, for example, be on one of the outermost layers of the wound dressing.

The wound dressing may comprise a release liner, which may, for example, be present on the skin-contacting side of the composition described herein. The release liner acts to facilitate ease of handling and packaging. The liner may, for example, assist in maintaining the wound dressing in a sterile condition. The release liner may, for example, be on one of the outermost sides of the wound dressing. The release liner may be any release liner known in the art that is compatible with the composition described herein. The release liner may, for example, comprise a silicone on the surface.

The wound dressing may, for example, contain an additional absorptive layer. The additional absorptive layer may, for example, be positioned between the adhesive and the backing. The additional absorptive layer may, for example, be selected from hydrophilic polyurethane foam, absorptive hydrogel, alginate fiber, superabsorbent sachet, and combinations thereof. In such construction the layer comprising the composition of the invention may be perforated.

The wound dressing may, for example, contain a carrier layer. The carrier lay may, for example, be a polyurethane film, a non-woven material, a hydrophobic foam, an ethylene vinyl acetate (EVA) film, or a polyethylene (PE) film. For example, the carrier may be a polyurethane film such as a Bioflex® Rx1281U or a Covestro® LPT9147T or a Covestro® LPT9147TX film. The carrier may, for example, be a polyethylene (PE) foam such as a Voltek Volara® foam, for example with nominal density of 4.0 lb/ft³ (0.064 g/cm³) and nominal thickness is 0.040 inches (0.1016 cm).

### Method of Making the Compositions and the Wound Dressings

There is also provided herein methods of manufacturing the compositions and wound dressings described herein.

The methods for making the compositions described herein may comprise mixing the acrylic adhesive, the thermoplastic absorbent polymer, and the gelling agent. The mixing may, for example, occur at a temperature equal to or greater than the melting point of the adhesive. The composition may, for example, be formed into a desirable shape and then cooled.

Each of the individual layers of the wound dressing may be made by methods known to those skilled in the art. The individual layers may be sourced separately and then adhered together in the desired form. Alternatively, one or more of the layers of the wound dressing may be made concurrently, for example in situ. Each of the layers may be adhered to the adjacent layer separately or all the layers in the wound dressing may be adhered to each other at once. The layers may, for example, be adhered to each other by application of heat and/or pressure.

### Uses of the Compositions and the Wound Dressings

The compositions and wound dressings described herein may be used for treating or preventing a wound. Thus, there is provided herein methods of treating or preventing a wound and compositions and wound dressings for use in treating or preventing a wound. Treating or preventing a wound comprises applying the composition and/or wound dressing to the wound.

The phrase “treating or preventing” refers to all forms of healthcare intended to remove or avoid the wound or to relieve its symptoms, including preventive and curative care, as judged according to any of the tests available according to the prevailing medical practice. An intervention that aims with reasonable expectation to achieve a particular result but does not always do so is included within the expression “treating or preventing”. An intervention that succeeds in accelerating progression of wound healing is included within the expression “treating or preventing”. The phrase “treating or preventing” in relation to a wound includes the initiation and/or maintenance and/or acceleration of all stages of wound healing (including hemostasis, inflammation, proliferation, maturation). However, the wound dressings described herein may be particularly used during stages of wound healing involving exudation of fluid from the wound.

The wound dressings described herein may be useful in treating or preventing various types of wound, primarily open wounds. The term wound includes skin cuts, grazes, abrasions, tears, burns, scalds, ulcers, spots, blisters and chafes, whether dermal, epidermal, or a combination of both. The wound may or may not be infected or contaminated. The wound may, for example, be chronic or acute. The wound may, for example, be a high-exudating wound.

The compositions and wound dressings described herein may be used for securing a device (e.g. a medical device) to skin (e.g., mammalian skin). Thus, there is provided herein methods of securing a device (e.g. a medical device) to skin (e.g. mammalian skin) and compositions and wound dressings for use in securing a device (e.g. a medical device) to skin (e.g. mammalian skin). Securing a device to the skin comprises applying the composition and/or wound dressing to the device and/or skin where the device is to be secured.

The compositions and wound dressings described herein may also be used for securing a device that has been inserted through the skin of a subject. Thus, there is provided herein methods of securing a device that is at least partially (e.g. fully) inserted in the skin of a subject and wound dressings for use in securing a device that is at least partially (e.g. fully) inserted in the skin of a subject. Securing the device comprises applying the composition and/or wound dressing to the area where the device has been inserted in the skin. The device may, for example, be a catheter. The device may, for example, be part of an ostomy system.

### EXAMPLES

### Materials

Acrylic hot melt adhesive HMA): Artimelt™ M11-1506 available from artimelt Inc., Tucker, Ga., USA.

Thermoplastic absorbent polymer (TAP): Tecophilic™ TG-2000 (thermoplastic polyurethane with a water absorption of 900%) available from Lubrizol, USA,

Gelling agent (GA): Carboxymethylcellulose (Aquasorb™ A500 available from Ashland, USA or Gelycel F1-600XX sodium carboxymethylcellulose available from Amtex, USA)

Non-adhesive coated polyurethane film: Bioflex® Rx1281U or Covestro® LPT9147T or Covestro® LPT9147TX carrier.

Polyethylene fibres: MiniFibers Inc. Short Stuff Synthetic Pulp E505F*

PE Foam: Voltek Volara® foam with nominal density of 4.0 lb/ft³ (0.064 g/cm³) and nominal thickness is 0.040 inches (0.1016 cm).

### Methods

The compositions specified in the Tables below were made as follows. The proportion of each component used is indicated in the results tables below.

### Examples 1 to 3

Using a z-blade mixer heated at 90° C. and using a mixing speed of between (30/50) to (50/83) rpm (Blade 1 Speed/Blade 2 Speed). The following procedure was followed. The acrylic hot melt adhesive was placed in the Z-blade mixer and mixed for 20 minutes to warm; the thermoplastic absorbent polymer was then added and mixed for 30 minutes. Once removed from the mixer the adhesive was pressed to a thickness of 0.3 mm and laminated with a polyurethane film (Bioflex® Rx1281U).

### Examples 4 to 5

A similar procedure to examples 1 to 3 was used. After the addition of the thermoplastic absorbent polymer, the mix was stirred for 20 mins and then the glycerol was added. The full composition was mixed for 30 mins before being removed from the mixer and being pressed using a hot press at 0.3 mm thickness and being laminated with a polyurethane film (Bioflex® Rx1281U).

### Examples 6 to 7

The thermoplastic absorbent polymer was placed in the mixer and mixed for 25 to 30 minutes at 90° C., after that the gelling agent was added. The mix was pressed to a 0.3 mm thickness using a hot press and then laminated with a polyurethane film (Bioflex® Rx1281U).

### Examples 8 to 13

A similar procedure to the above examples was used. In a Z-blade mixer at 90° C., the mixing order and mixing time at each step was as follows:
1—Hot melt (mixed for 20 minutes)
2—Glycerol (mixed for 25 minutes)
3—Carboxyl Methyl Cellulose (mixed for 30 minutes)

The mix was pressed to a 0.3 mm thickness using a hot press and then laminated with a polyurethane film (Bioflex® Rx1281U).

### Examples 14 to 17

A similar procedure to the above examples was used. In a Z-blade mixer at 90° C., the mixing order and mixing time at each step was as follows:
1—Hot melt (mixed for 20 minutes)
2—TG2000 (mixed for 25-30 minutes)
3—Carboxyl Methyl Cellulose (mixed for 30 minutes)

The mix was pressed to a 0.3 mm thickness using a hot press and then laminated with a polyurethane film (Bioflex® Rx1281U).

### Examples 18 to 33

A similar procedure to the above examples was used. In a Z-blade mixer at 90° C., the mixing order and mixing time at each step was as follows:
1—Hot melt (mix for 20 minutes)
2—TG2000 (mixed for 25-30 minutes)
3—Glycerol (mixed for 25 minutes)
4—Carboxyl Methyl Cellulose (mixed for 30 minutes)

The mix was pressed to a 0.3 mm thickness using a hot press and then laminated with a polyurethane film (Bioflex Rx1281U).

The moisture vapour transmission rate (MVTR), absorption, tack, and peel of each composition was measured as follows.

### Fluid Handling Capacity Test

The ISO13726 is used to measure the fluid handling capacity, which is the sum of the absorption and moisture vapor transmission rate. To perform this test first a saline solution is prepared by dissolving 34.2 g of NaCl into 3.79 litres of distilled water. A circular disc having a diameter of 5.08 cm (2″) of the test article is cut (thickness of 0.3 mm). The release liner is then removed and the sample placed on a paddington cup. The paddington cup and the sample is weighted (W₁). The paddington cup is filled with 20 ml of saline solution and closed, the total construction is then weighed again (W₂). The cups are then placed in an oven containing dry silica beads at 37° C. for 24 hours. After 24 hours the cups are removed from the oven and are allowed to equilibrate at room temperature for 20 minutes. The cup assembly is then weighed (W₃). The cup is opened to remove the bottom cover and empty the saline solution. The cup is then inverted for 15 to 20 minutes to dry and is weighed (W₄). MVTR and absorption is calculated as follows.

MVTR=W₂−W₃

Absorption=W₄−W₁

Fluid Handling Capacity=Weight of Moisture Vapour Lost+Weight of Fluid Absorbed=(W₂−W₃)+(W₄−W₁)

### Tack Test

The tack test method is used to determine the tack properties of a pressure sensitive adhesive. Tack force measures the force required to separate the adhesive from a surface shortly after they have been brought into contact under a load equal to the weight of a pressure sensitive article.

The test composition is shaped in a circular 2.54 cm (1″) diameter and is tested using a Calibrated PT-1000 Probe Tack Tester. The test composition is placed on the annular ring in the Test Platform. The area placed over the test hole should be free from any indentation caused by either the release liner cut or the z-fold. The test area should be smooth. The testing probe (diameter 5 mm) and the Annular Ring (applies force of 9.79±0.1 kPa) are cleaned with a lint-free wipe containing alcohol and allowed to dry for 10 minutes. The testing cycle is then initiated—the test platform moves downward and maintains a probe contact for a dwell time of 1 second. The test platform then moves upward at a rate of 60.96 cm/minute (24 inch/min). The peak force to remove the probe from the adhesive is recorded and corresponds to the tack.

### Peel Test

The peel test measures the force required to remove a pressure sensitive tape from a stainless steel 304 test panel at a 90° angle and at a standard rate of 5.0 mm/s in accordance with ASTM A 666. The test composition is cut in a 25 mm (±1.5 mm) width and 100 mm (±1.5 mm) length shape. The test composition and test panel are conditioned at room temperature for 8 hours. The test panels are 5 mm (±1.5 mm) by 15 mm (±1.5 mm) and are cleaned with acetone after each use and allowed to dry for 5 minutes.

At one end on the sample 20-30 mm of the adhesive is carefully separated from its protective liner, which will later be affixed to the upper clamps. The entire specimen is removed from its liner and placed onto the test panel. The specimen should fall gently onto the panel, then using only the weight 2.64 kg (±0.1 kg) of the roller, the adhesive sample is applied to the panel by rolling once forward and once backward in each lengthwise direction. The test composition is affixed to the tensile tester and the sample is peeled. The average of the peel force is calculated by removing the first 5% of the beginning and end of the sample (Speed of pulling is 300 mm/min and data recorded at approximately 3.3 Hz).

### Results

TABLE 1
Compositions comprising acrylic hot melt adhesive and thermoplastic adsorbent polymer

| | | | | GA | GA | MVTR | Absorption | Tack | Peel |
|---|---|---|---|---|---|---|---|---|---|
| | HMA | Glycerol | TAP | (A500) | (Gelycel) | (g/m²*24 hrs) | (g/10 cm²) | (g) | (N) |
| Example 1 | 0.10 | — | 0.90 | — | — | — | — | 0 | 0.00 |
| Example 2 | 0.70 | — | 0.30 | — | — | 293 | 0.52 | 183 | 2.56 |
| Example 3 | 0.90 | — | 0.10 | — | — | 391 | 0.41 | 166 | 2.81 |
| Example 4 | 0.70 | 0.09 | 0.21 | — | — | 493 | 0.39 | 203 | 2.67 |
| Example 5 | 0.70 | 0.09 | 0.21 | — | — | 632 | 0.19 | 150 | 1.93 |

The melt flow index (MFI) of the thermoplastic absorbent polymer used in each composition was as follows:
Example 1: 20.1 g/10 minutes
Examples2 and 4: 6.8 g/10 minutes
Example 3: 20.1 g/10 minutes
Example 5: 20.1 g/10 minutes

Compositions comprising acrylic hot melt adhesive and thermoplastic absorbent polymer without a gelling agent have low absorption and low MVTR, which makes these compositions inappropriate for use in wound care or ostomy.

TABLE 2
Compositions comprising thermoplastic absorbent polymer and gelling agent

| | | | | GA | GA | MVTR | Absorption | Tack | Peel |
|---|---|---|---|---|---|---|---|---|---|
| | HMA | Glycerol | TAP | (A500) | (Gelycel) | (g/m²*24 hrs) | (g/10 cm²) | (g) | (N) |
| Example 6 | — | — | 0.70 | — | 0.30 | — | — | 0.00 | 000 |
| Example 7 | — | — | 0.50 | — | 0.50 | — | — | 0.00 | 000 |

The melt flow index (MFI) of the thermoplastic absorbent polymer used in each composition was as follows:
Example 6: 20.1 g/10 minutes
Example 7: 20.1 g/10 minutes

Compositions comprising thermoplastic absorbent polymer and gelling agent without an acrylic hot melt adhesive have low tack and peel, which makes these compositions inappropriate for adhering to mammalian skin.

TABLE 3
Compositions comprising acrylic hot melt adhesive and gelling agent

| | | | | GA | GA | MVTR | Absorption | Tack | Peel |
|---|---|---|---|---|---|---|---|---|---|
| | HMA | Glycerol | TAP | (A500) | (Gelycel) | (g/m²*24 hrs) | (g/10 cm²) | (g) | (N) |
| Example 8 | 0.70 | — | — | 0.30 | — | 1048 | 4.10 | 111 | 2.38 |
| Example 9 | 0.60 | — | — | — | 0.40 | 823 | 8.30 | 116 | 2.99 |
| Example 10 | 0.80 | — | — | — | 0.20 | 447 | 0.75 | 173 | 2.82 |
| Example 11 | 0.70 | — | — | — | 0.30 | 84 | 4.63 | 142 | 2.72 |
| Example 12 | 0.70 | 0.05 | — | 0.25 | — | 755 | 4.06 | 137 | 2.94 |
| Example 13 | 0.70 | 0.10 | — | 0.20 | — | 923 | 2.43 | 143 | 2.48 |

The melt flow index (MFI) of the thermoplastic absorbent polymer used in each composition (Examples 8 to 13) was 6.8 g/10 minutes.

Compositions comprising acrylic hot melt adhesive and gelling agent without thermoplastic absorbent polymer have low tack, which makes these compositions inappropriate for adhering to mammalian skin. Example 10 had good adhesion but lacked good absorption properties.

TABLE 4
Compositions comprising acrylic hot melt adhesive,
thermoplastic absorbent polymer, and gelling agent

| | | | | GA | GA | MVTR | Absorption | Tack | Peel |
|---|---|---|---|---|---|---|---|---|---|
| | HMA | Glycerol | TAP | (A500) | (Gelycel) | (g/m²*24 hrs) | (g/10 cm²) | (g) | (N) |
| Example 14 | 0.725 | — | 0.03 | — | 0.25 | 663 | 4.51 | 172 | 2.63 |
| Example 15 | 0.70 | — | 0.025 | — | 0.275 | 734 | 5.22 | 166 | 2.66 |
| Example 16 | 0.70 | — | 0.15 | 0.15 | — | 773 | 4.77 | 170 | 2.48 |
| Example 17 | 0.10 | — | 0.70 | — | 0.20 | — | — | 0 | 0.00 |

The melt flow index (MFI) of the thermoplastic absorbent polymer used in each composition (Examples 14 to 17) was 6.8 g/10 minutes.

Compositions comprising acrylic hot melt adhesive, thermoplastic absorbent polymer and gelling agent have good absorption and good tack. If the amount of thermoplastic absorbent polymer is high compared to the acrylic hot melt adhesive, adhesion becomes negligible (example 17).

TABLE 5
Compositions comprising acrylic hot melt adhesive, thermoplastic
absorbent polymer, gelling agent, and glycerol

| | | | | GA | GA | MVTR | Absorption | Tack | Peel |
|---|---|---|---|---|---|---|---|---|---|
| | HMA | Glycerol | TAP | (A500) | (Gelycel) | (g/m²*24 hrs) | (g/10 cm²) | (g) | (N) |
| Example 18 | 0.70 | 0.09 | 0.16 | 0.05 | — | 715 | 2.65 | 136 | 2.39 |
| Example 19 | 0.70 | 0.08 | 0.05 | 0.17 | — | 877 | 4.67 | 155 | 2.48 |
| Example 20 | 0.70 | 0.08 | 0.05 | 0.17 | — | 938 | 3.79 | 158 | 2.54 |
| Example 21 | 0.70 | 0.08 | 0.02 | 0.20 | — | 848 | 3.52 | 153 | 3.00 |
| Example 22 | 0.70 | 0.03 | 0.10 | 0.18 | — | 740 | 4.17 | 144 | 2.30 |
| Example 23 | 0.70 | 0.0255 | 0.0255 | 0.25 | — | 759 | 4.58 | 163 | 2.58 |
| Example 24 | 0.70 | 0.0255 | 0.0255 | — | 0.25 | 869 | 5.23 | 164 | 2.60 |
| Example 25 | 0.70 | 0.08 | 0.05 | — | 0.17 | 952 | 3.03 | 178 | 2.97 |
| Example 26 | 0.67 | 0.12 | 0.06 | — | 0.15 | 802 | 3.75 | 149 | 2.94 |
| Example 27 | 0.69 | 0.03 | 0.01 | — | 0.28 | 763 | 4.16 | 160 | 2.96 |
| Example 28 | 0.70 | 0.0255 | 0.0255 | — | 0.25 | 778 | 4.95 | 176 | 3.09 |
| Example 29 | 0.14 | 0.12 | 0.60 | — | 0.14 | — | — | 0 | 0.00 |
| Example 30 | 0.70 | 0.03 | 0.03 | — | 0.25 | 769 | 5.17 | 165 | 2.37 |
| Example 31 | 0.70 | 0.0250 | 0.0250 | — | 0.25 | 786 | 4.41 | 185 | 2.45 |

The melt flow index (MFI) of the thermoplastic absorbent polymersed in the compositions Examples 18 to 29) was 6.8 g/10 minutes.

The melt flow index (MFI) of the thermoplastic absorbent polymer used Example 30 was 21.4 g/10 minutes.

The melt flow index (MFI) of the thermoplastic absorbent polymer used Example 31 was 5.8 g/10 minutes.

Compositions additionally comprising glycerol also have good absorption and tack, as well as the benefit of addition a cost-effective compound which is known to have skin moisturizing properties.

Examples 30 and 31 demonstrate that, even with thermoplastic polymers having different melt flow indexes, good absorption and tack can be obtained.

TABLE 6
Compositions comprising acrylic hot melt adhesive, thermoplastic absorbent
polymer, gelling agent, glycerol, and polyethylene fibres

| | | | | GA | GA | MVTR | Absorption | Tack | Peel |
|---|---|---|---|---|---|---|---|---|---|
| | HMA | Glycerol | TAP | (A500) | (Gelycel) | (g/m²*24 hrs) | (g/10 cm²) | (g) | (N) |
| Example 32 | 0.70 | 0.03 | 0.03 | 0.02 | 0.23 | 592 | 4.13 | 123 | 2.88 |
| Example 33 | 0.69 | 0.05 | 0.01 | 0.02 | 0.23 | 638 | 3.80 | 143 | 2.30 |
| Example 34 | 0.69 | 0.0500 | 0.0500 | 0.01 | 0.20 | 673 | 4.74 | 124 | 2.27 |
| Example 35 | 0.70 | 0.05 | 0.01 | 0.01 | 0.23 | 769 | 4.10 | 133 | NT |
| Example 36 | 0.69 | 0.0250 | 0.5000 | 0.02 | 0.22 | 723 | 4.56 | 123 | NT |
| Example 37 | 0.70 | 0.03 | 0.03 | 0.01 | 0.24 | 659 | 4.54 | 152 | 2.30 |

The melt flow index (MFI) of the thermoplastic absorbent polymer used in Examples 32 to 37 was 20.1 g/10 minutes,

Compositions additionally comprising polyethylene fibres have improved repositionability and fluid handling capacity. They also assist in maintaining the integral structure of the hydrocolloid adhesive when in contact with fluids with little effect on tack.

TABLE 7
Compositions comprising acrylic hot melt adhesive, thermoplastic
absorbent polymer, gelling agent, and glycerol with a carrier

| | | | | | GA | GA | MVTR | Absorption |
|---|---|---|---|---|---|---|---|---|
| | Carrier | HMA | Glycerol | TAP | (A500) | (Gelycel) | (g/m²*24 hrs) | (g/10 cm²) |
| Example 38 | Bioflex ® Rx1281U | 0.70 | 0.0255 | 0.0255 | — | 0.25 | 869 | 5.23 |
| Example 39 | Covestro ® LPT9147TX | 0.70 | 0.0255 | 0.0255 | — | 0.25 | 5873 | 2.46 |
| Example 40 | Covestro ® LPT9147TX | 0.70 | 0.0255 | 0.0255 | — | 0.25 | 5998 | 2.72 |
| Example 41 | PE Foam - Voltek Volara ®* | 0.70 | 0.0255 | 0.0255 | — | 0.25 | 306 | 4.13 |

*The selected Voltek Volara had a normal density of 4.0 lb/ft³ (0.064 g/cm³) and nominal thickness of 0.040 inches (0.1016 cm).

The melt flow index (MFI) of the thermoplastic absorbent polymer used Examples 38 to 41 was 20.1 g/10 minutes.

Compositions having different carriers also provide good fluid handling capacity.

The foregoing broadly describes certain embodiments of the present invention without limitation. Variations and modifications as will be readily apparent to those skilled in the art are intended to be within the scope of the present invention as defined in and by the appended claims.

1. A composition comprising an acrylic adhesive, a thermoplastic absorbent polymer, and a gelling agent.

2. The composition of claim 1, wherein the acrylic adhesive is an acrylic hot melt adhesive.

3. The composition of any preceding claim, wherein the composition comprises:
at least about 40 wt % of an acrylic adhesive;
at least about 1 wt % of a thermoplastic absorbent polymer; and
at least about 1 wt % of a gelling agent.

4. The composition of any preceding claim, wherein the composition comprises:
up to about 98 wt % of an acrylic adhesive; and/or
up to about 20 wt % of a thermoplastic absorbent polymer; and/or
up to about 50 wt % of a gelling agent.

5. The composition of any preceding claim, wherein the composition comprises from about 60 wt % to about 80 wt % of an acrylic adhesive, for example wherein the acrylic adhesive is an acrylic hot melt adhesive.

6. The composition of any preceding claim, wherein the composition comprises from about 1 wt % to about 10 wt % of a thermoplastic absorbent polymer.

7. The composition of any preceding claim, wherein the composition comprises from about 15 wt % to about 30 wt % of a gelling agent.

8. The composition of any preceding claim, wherein the thermoplastic absorbent polymer absorbs water in an amount of at least about 100% of its own weight.

9. The composition of any preceding claim, wherein the thermoplastic absorbent polymer has a glass transition temperature (Tg) equal to or less than about 0° C.

10. The composition of any preceding claim, wherein the thermoplastic absorbent polymer has a melting temperature equal to or less than about 120° C.

11. The composition of any preceding claim, wherein the thermoplastic absorbent polymer is a polyurethane polymer.

12. The composition of any preceding claim, wherein the gelling agent is a superabsorbent polymer or a hydrocolloid.

13. The composition of any preceding claim, wherein the gelling agent is carboxymethylcellulose (CMC), for example sodium carboxymethylcellulose.

14. The composition of any preceding claim, wherein the composition further comprises a polyol such as glycerol.

15. The composition of any preceding claim, wherein the composition further comprises an active agent.

16. The composition of any preceding claim, wherein the composition further comprises fibres such as polyethylene fibres.

17. The composition of any preceding claim, wherein the composition has a thickness ranging from about 100 μm to about 2000 μm, for example from about 200μm to about 500 μm.

18. A wound dressing comprising a composition of any preceding claim.

19. The wound dressing of claim 18, wherein the wound dressing further comprises a breathable backing such as a breathable film.

20. The wound dressing of claim 19, wherein the breathable backing is a polyether-polyurethane copolymer film.

21. A method of making a composition of any of claims 1 to 17, the method comprising mixing the acrylic adhesive, the thermoplastic absorbent polymer, and the gelling agent.

22. A composition or wound dressing of any preceding claim for treating or preventing a wound and/or for securing a device to skin.

23. Use of a composition or wound dressing of any preceding claim for treating or preventing a wound and/or for securing device to skin.

24. A method of treating a wound, the method comprising applying a composition or wound dressing of any preceding claim to the wound.

25. A method of securing a device to skin, the method comprising applying a composition or wound dressing of any preceding claim to the device and/or skin where the device is to be secured.

26. The composition or wound dressing for use of claim 22, the use of claim 23, or the method of claim 24 or 25, wherein the wound is a chronic wound or an acute wound.

27. The composition or wound dressing for use of claim 22, the use of claim 23, or the method of claim 24 or 25, wherein the device is part of a catheter or ostomy system.
